(19) United States
(12) Patent Application Publication
(10) Pub. No.: US 2008/0187093 A1
(43) Pub. Date: Aug. 7, 2008

(54) X-RAY GENERATION USING SECONDARY EMISSION ELECTRON SOURCE

(76) Inventors: John Scott Price, Niskayuna, NY (US); Loucas Tsakalakos, Niskayuna, NY (US); Pierre-Andre Bui, Clifton Park, NY (US); Bruce Matthew Dunham, Ithaca, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/671,878

(22) Filed: Feb. 6, 2007

(51) Int. Cl. H01J 35/06; A61B 6/03

(52) U.S. Cl. 378/19; 378/136; 313/346 R

(57) ABSTRACT

A method and apparatus are provided for generating high frequency electromagnetic energy using a secondary emission electron source. An x-ray source is therefore provided having a primary electron emitter, a secondary emission member, and an anode. The primary electron emitter provides a primary electron current directed to the secondary emission member. The secondary emission member then generates a secondary electron current which causes x-ray generation when impinging upon the anode.

### BACKGROUND OF THE INVENTION

The present invention relates generally to the generation of high frequency electromagnetic energy and, more particularly, to a method and apparatus of using a secondary electron emission member in providing an electron stream used to generate x-rays.

X-ray generating systems typically include an electron generating cathode and an anode in a sealed housing. The cathode provides an electron stream or current that is directed toward the anode. Many x-ray tubes include a rotating anode structure for distributing the heat generated at a focal spot. The anode is typically rotated by an induction motor having a cylindrical rotor built into a cantilevered axle. The axle supports a disc-shaped anode target as well as an iron stator structure with copper windings that surrounds an elongated neck of the x-ray tube. The rotor of the rotating anode assembly is driven by the stator. An x-ray tube cathode provides a focused electron beam that is accelerated across an anode-to-cathode vacuum gap and produces x-rays upon impact with the anode. Because of the high temperatures generated when the electron beam strikes the target, it is desirable to rotate the anode assembly at high rotational speed.

One particular use of such x-ray generators is in the field of diagnostic imaging. Typically, in computed tomography (CT) imaging systems, for example, an x-ray source emits a fan-shaped beam toward a subject or object, such as a patient or a piece of luggage. The beam, after being attenuated by the subject, impinges upon an array of radiation detectors. The intensity of the attenuated beam radiation received at the detector array is typically dependent upon the attenuation of the x-ray beam by the subject. Each detector element of the detector array produces a separate electrical signal indicative of the attenuated beam received by each detector element. The electrical signals are transmitted to a data processing system for analysis which ultimately produces an image.

Generally, the x-ray tube or generator and the detector array are rotated about the gantry within an imaging plane and around the subject. X-ray detectors typically include a collimator for collimating x-ray beams received at the detector, a scintillator for converting x-rays to light energy adjacent the collimator, and photodiodes for receiving the light energy from the adjacent scintillator and producing electrical signals therefrom. Typically, each scintillator of a scintillator array converts x-rays to light energy. Each scintillator discharges light energy to a photodiode adjacent thereto. Each photodiode detects the light energy and generates a corresponding electrical signal. The outputs of the photodiodes are then transmitted to the data processing system for image reconstruction.

In order to generate an x-ray beam of sufficient strength for CT and other x-ray based diagnostic imaging modalities, cathode assemblies of x-ray tubes often provide close to 1 amp of electron current. The electrons emitted from a cathode are accelerated across the vacuum gap of the x-ray tube to the anode by voltages on the order of 20 to 150 kVp. To achieve electron emission from a thermionic emitter, for example, a control voltage of about 10 V is applied across the tungsten filament, producing high temperatures and a current of about 7 amps in the filament. Therefore, adjustments to the cathode control voltage and/or current regulate the tube current.

The high voltage vacuum environment within many x-ray tubes presents additional considerations for cathode design. Some attempts to reduce the power demands of an x-ray tube cathode have utilized specially designed materials having lower work functions than ordinary thermionic filaments. Others have sought to incorporate field emitter arrays (FEAs) into cathode assemblies. However, the harsh environment of an x-ray tube can reduce the efficiency over time and limit the lifespan of such emitters. Thus, these emitters may not be robust enough for use in x-ray tubes due to their chemical, electrical, and physical sensitivity as well as other effects arising from back-bombarding ions, uncontrolled voltages and currents from vacuum arcing, and other particle and radiation exposures.

Therefore, it would be desirable to have an apparatus and method for generating x-rays useable in diagnostic imaging which overcome the aforementioned drawbacks. In particular, it would be desirable to reduce the power and temperature requirements of cathode assemblies for x-ray generators while maintaining a durability to survive a high voltage vacuum environment.

### BRIEF DESCRIPTION OF THE INVENTION

The present invention is directed to a method and apparatus for generating x-rays via x-ray tubes, or other x-ray generators, having a secondary emission cathode. By utilizing a secondary emission member in electron stream production, the primary electron emitter of a cathode can operate more efficiently and can be protected from harsh operating environments.

In accordance with one aspect of the present invention, an x-ray generator includes a primary electron source, a controller, a secondary emission component, and an anode. The controller is configured to apply an electrical potential to the primary electron source so as to cause it to emit a primary stream of electrons. The secondary emission component is positioned in the path of this primary stream of electrons. When the primary stream of electrons strikes the secondary emission component, the secondary emission component emits a secondary stream of electrons. The anode is configured to emit x-rays when the secondary stream of electrons strikes the anode.

According to another aspect of the present invention, a cathode assembly is provided which has at least one electron emitting member, a secondary emission member, and a controller. The electron emitting member(s) has two ends, the first end being configured for electron emission. The secondary emission member is positioned over the first end of the electron emitting member and is separated therefrom. When the controller applies a first voltage to the electron emitting member, an electron current is generated by the first end of the electron emitting member. The secondary emission member amplifies this electron current such that it becomes sufficient for generation of x-ray beams.

In another aspect of the present invention, an x-ray tube for an imaging system is provided. The x-ray tube has a housing that encloses an anode and a cathode. The cathode includes a primary electron emission member and a secondary electron emission member which shields the primary electron emission member. The anode is positioned in an electron path of the cathode and is configured to emit a beam of high-frequency electromagnetic energy when a stream of electrons from the cathode impinges thereon. The beam of high-frequency electromagnetic energy is conditioned for use in a CT imaging process.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a cross-sectional view of a field emitter electron source in accordance with an embodiment of the present invention.

FIG. 2 is a perspective view of an array-type electron source, in partial cut-away, in accordance with an embodiment of the present invention.

FIG. 3 is a cross-sectional view of a field emitter electron source in accordance with another embodiment of the present invention.

FIG. 4 is a cross-sectional view of a field emitter electron source in accordance with a further embodiment of the present invention.

FIG. 5 is a cross-sectional view of a thermionic electron source in accordance with an embodiment of the present invention.

FIG. 6 is a cross-sectional view of a ferro-electric electron source in accordance with an embodiment of the present invention.

FIG. 7 is a schematic view of an x-ray source in accordance with an embodiment of the present invention.

FIG. 8 is a schematic view of an x-ray source in accordance with another embodiment of the present invention.

FIG. 9 is a perspective view of a CT imaging system incorporating an embodiment of the present invention

FIG. 10 is a schematic block diagram of the system illustrated in FIG. 9.

FIG. 11 is a perspective view of a CT system for use with a non-invasive package inspection system in accordance with another embodiment of the present invention.

### DETAILED DESCRIPTION OF THE PREFERRED EMBODIMENT

One operating environment for the present invention is described with respect to a sixty-four-slice “third generation” computed tomography (CT) system. However, it will be appreciated by those skilled in the art that the present invention is equally applicable for use with other imaging modalities, such as x-ray projection imaging, package inspection systems, as well as other multi-slice CT configurations or systems. Moreover, the present invention will be described with respect to the generation, detection and/or conversion of x-rays. However, one skilled in the art will further appreciate that the present invention is also applicable for the generation, detection, and/or conversion of other high frequency electromagnetic energy.

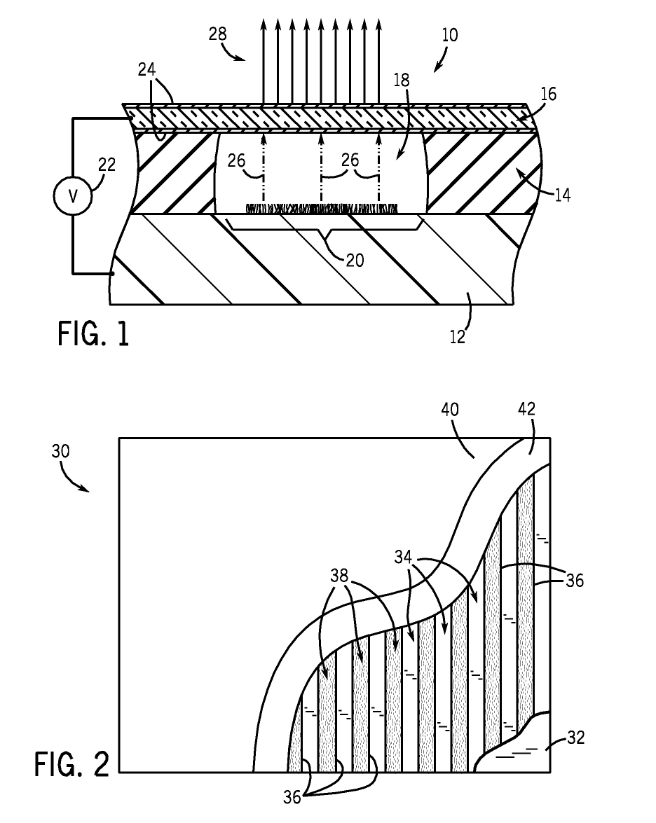

Referring to FIG. 1, a cross-sectional view of an electron source 10 is depicted. As shown, electron source 10 is a field emitter array; though it is appreciated and will be shown below that other types of electron sources may also incorporate features and advantages of the present invention. Electron source 10 has a base or substrate layer 12, preferably formed of a conductive or semiconductive material such as a silicon-based substance. Therefore, substrate layer 12 is preferably rigid and opaque. An insulating layer 14 is deposited over substrate 12, to separate a secondary emission layer 16 therefrom. Insulating layer 14 is preferably formed of SiO₂ or some other material having similar dielectric properties. A channel or aperture 18 is formed in insulating layer 14, by any of several known chemical or etching manufacturing processes.

Secondary emission layer 16 is disposed over insulating layer 14 and covers the channel 18 thereof. Secondary emission layer 16 may be formed of a thin sheet or film of some secondary-electron emissive material which produces a yield of secondary electrons upon impact of a primary electron thereupon. In general, any materials which have a high secondary electron yield may be suitable. Such materials are typically characterized by having a highly negative electron affinity (NEA). One substance particularly advantageous for use in secondary emission is diamond. Diamond and other diamond-like substances have a high secondary electron yield, are relatively resistant to chemical contamination, and operate efficiently, with low dark noise at high temperatures. It is appreciated, however, that secondary emission layer 16 may take on several other structural formations (crystalline, polycrystalline, or other forms), may be oriented or shaped differently with respect to substrate 12, and may be formed of multiple other materials (such as AlN, BN, GalAlN-based substances, and AlNSiC-based substances). The secondary emission layer 16 may also be doped with p- or n-type dopants to improve the secondary emission yield. Similarly, in instances in which a high amplification of a primary electron current is desirable, a number of secondary emission layers 16 may be overlapped or otherwise placed in series to provide an additional secondary electron yield to electron source 10.

A plurality of emitters 20 are formed on substrate 12 in channel 18. As shown, emitters 20 are carbon nanotube emitters, though it is appreciated that other emitters are possible. For example, a layer of some substance having a low work function or high NEA could be substituted for or used in combination with nanotube emitters 20. Alternatively, inorganic or metallic nanowires could also be utilized in place of, or in conjunction with emitters 20. A controller 22 is connected to supply a voltage between substrate 12 and secondary emission layer 16. Secondary emission layer 16 is therefore coated or covered with a very thin metallic layer 24. As shown, metal coating 24 covers both the top and bottom surfaces of secondary emission layer 16, though it is appreciated that metal coating 24 may cover only one surface, such as the “underside” or primary electron side. Alternatively, coating 24 may be lithographed or printed in a specific pattern on secondary emission layer 16. For example, metal coating 24 may be deposited on secondary emission layer 16 such that it does not cover, hinder, or obstruct electron current therethrough or such that it disperses arcing events.

The electric field caused by the controller voltage 22 induces a primary electron stream 26 to be emitted from emitters 20. The primary electron stream 26 is accelerated across channel 18 by the difference in electrical potential and impinges upon secondary emission layer 16. In this regard, channel 18 is preferably a vacuum gap and metal coating 24 is preferably thin enough to pass electron stream 26 therethrough. When struck by primary electron current 26, secondary emission layer 16 amplifies the stream 26 and emits a secondary electron stream 28 stronger than primary electron stream 26. In embodiments in which secondary emission layer 16 is a diamond film, secondary electron stream 28 may be an electron current 10 to 100 times stronger than that of primary electron stream 26.

FIG. 2 shows an upper perspective view of an array 30 of the electron source of FIG. 1. The array 30 of FIG. 2 has a substrate or base 32, visible in cut-away, and an insulating layer 34 formed thereon. As shown, the substrate 32 is common to all emitters 38 of the array 30. A number of channels 36 are formed in the insulating layer 34 and are filled with rows of emitters 38. A secondary emission layer 40 is shown in partial cut-away, and covers the entire array 30. As discussed above, secondary emission layer 40 is preferably a substance having a high NEA and may be covered or partially covered with a metal coating 42. In manufacture, array 30 and the rows of emitters 38 thereof may be relatively small. That is, field emitter arrays are scalable down to a few millimeters in size and emitters are on the order of micrometers in size.

As shown, secondary emission layer 40 covers all emitters 38 of the array, providing shielding and protection to the emitters 38 from back-bombarding ions, electric arcs, and other phenomena associated with environments such as x-ray tubes. However, it is appreciated that secondary emission layer 40 may also provide shielding from other effects, such as chemical contamination and physical damage associated with other uses of array 30. While providing such shielding to emitters 38, secondary emission layer 40 may simultaneously operate as the gate electrode for all emitters 38 of array 30 to cause electron emission from the emitters 38. It is understood that the thickness of secondary emission layer 40 may vary from 0.01 micrometers to hundreds of micrometers, thinner layers providing better secondary electron yield and thicker layers providing improved shielding.

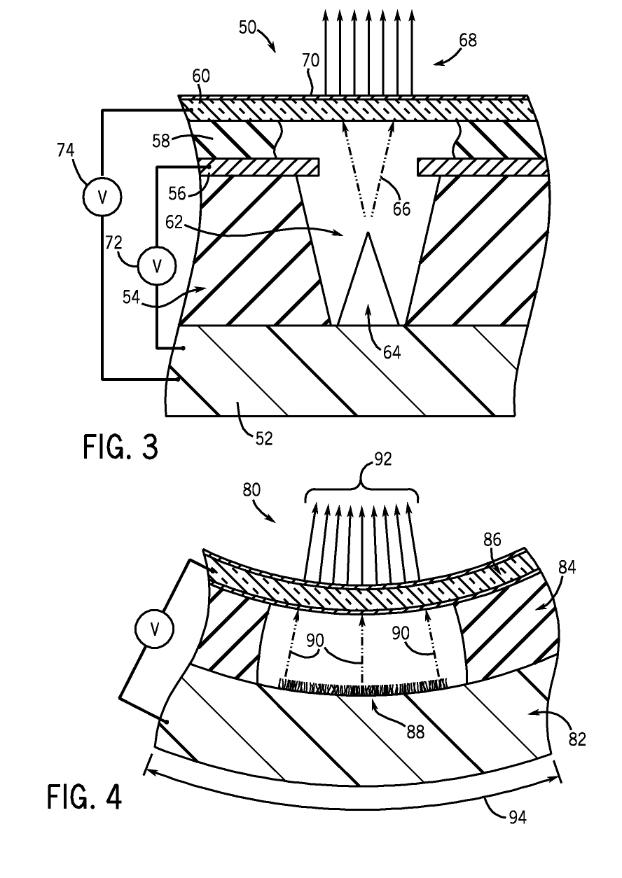

FIG. 3 shows a cross-section of an alternative embodiment of the present invention incorporating what is known as “Spindt”-type electron emission. Electron source 50 includes a conductive, opaque substrate 52, a first insulating layer 54, an electrode or gate layer 56, a second insulating layer 58, and a secondary emission layer 60. An opening or aperture 62 is formed in first insulating layer 54, through gate layer 56 and second insulating layer 58. A cone-type emitter tip 64 is positioned in aperture 62 and oriented such that electrons 66 emitted therefrom are directed toward secondary emission layer 60. When grouped as a field-emitter array (not shown), emitters such as shown in FIG. 3 will typically have common substrate, insulating, and gate layers, though each emitter will be housed in an individual aperture or opening.

Emitter tip 64 is a Spindt-type emitter, and in some embodiments, may be formed of molybdenum metal, though it is appreciated that other substances and coatings are also useable. For example, emitter tip 64 may be coated with low work function mixed oxide particles. When a primary emission voltage, representationally depicted as voltage source 72, is applied, an electric field at electrode or gate layer 56 causes emitter tip 64 to emit a primary stream of electrons. Gate layer 56 accelerates the stream of electrons 66 across the aperture 62. In this regard, aperture 62 may be a vacuum gap. A secondary emission voltage 74 is applied between the substrate 52 and a conductive coating, or metal layer 70 of secondary emission layer 60. When the secondary emission voltage 74 is being applied to secondary emission layer 60 and the primary stream of electrons 66 impinges thereupon, secondary emission layer 60 emits a secondary stream of electrons 68, stronger than the primary stream of electrons. Thus, secondary emission layer 60 is physically and electrically separate from the gate layer 56. It is appreciated that such a configuration is also useable with the other configurations and embodiments described herein, such as the nanotube-emitter embodiment depicted in FIGS. 1 and 2.

FIG. 4 shows another alternative embodiment of the present invention, in which an electron source 80 is configured to focus the electron emissions therefrom. Electron source 80 is depicted in a partial cross-sectional view to illustrate a curvature 94 thereof. As shown, a substrate layer 82, an insulating layer 84, and a secondary emission layer 86 are curved such that a primary electron current 90 from emitters 88 and a secondary electron current 86 from secondary emission layer 86 tend to converge. Preferably, curvature 94 may be concave and chosen to cause a desired convergence or focusing for a particular distance, such as cathode to anode electron acceleration distance of an x-ray tube (discussed below). As known in the art, varying the area of the anode on which an electron current impinges varies characteristics of the resulting x-ray beam. Alternatively, some embodiments of the present invention may have only a curved substrate 82 to focus the primary electron current 90 and other embodiments may have only a curved secondary emission layer 86 to focus the secondary electron current 92. Furthermore, it is understood that, while only a single row of emitters 88 is shown, curvature 94 may extend across multiple rows of emitters in a field emitter array (not shown) and that such an array may be curved across more than one dimension.

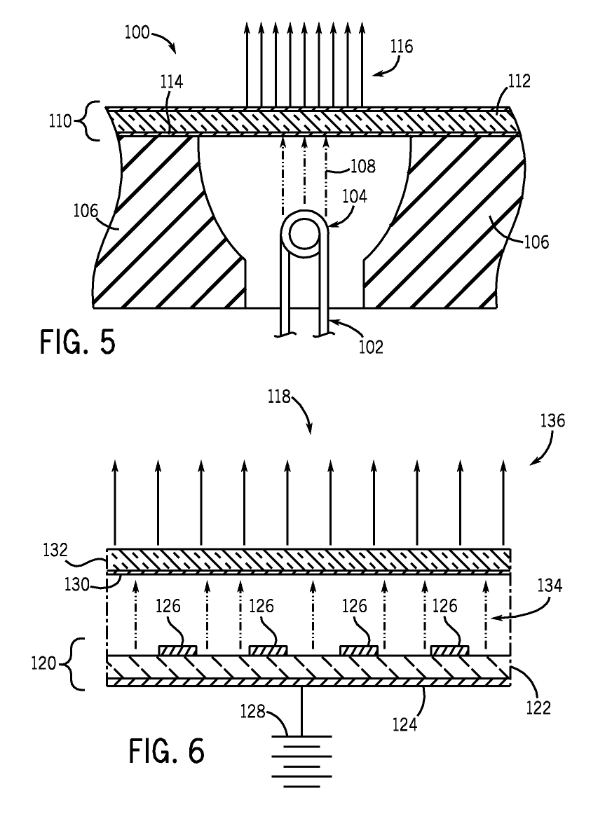

FIG. 5 depicts another embodiment of the present invention in which the primary electron source of an electron generator 100 is a thermionic emission filament 102. As known in the art, thermionic filaments 102 emit electrons when a high current flows therethrough, increasing the temperature of the filament 102. Thus, filaments are generally shaped to have a coil portion 104 to maximize electron emission within the electron generator 100. Typically, thermionic filaments are formed of tungsten, though other materials and combinations of materials are possible. Tungsten filament emitters generally have a work function of about 4.5 eV, though the filaments can be combined or substituted with materials having a lower work function, such as 3.0 eV and below. For example, tungsten filaments may be coated with low work function mixed oxide particles.

Thermionic filaments 102 are customarily housed in an insulator or focusing cup 106, to direct the primary electron current 108 out of the electron generator in a desired direction, as shown. Therefore, a secondary emission layer 110 may be placed over the focusing cup 106 to provide a secondary electron current 116 when struck by the primary electron current 108. As discussed above, a secondary emission layer 110 may be formed of diamond 112, a diamond-like substance, or another material having a high NEA. With the incorporation of secondary emission layer 110, a current applied to filament 102 may be less than would normally be required to produce a desired x-ray beam intensity. In other words, a primary electron current 108 of the filament 102 may be itself insufficient to produce the desired x-ray beam intensity. The secondary electron current 116, however, will be sufficient to produce the desired x-ray beam intensity.

Secondary emission layer 110 may also have a conductive or metal coating or partial-coating 114. Thus, a control voltage may be applied to accelerate the primary electron current 108 towards the secondary emission layer 110. In other embodiments, secondary emission layer may lack a metal coating or may be positioned differently with respect to filament 102. For example, in some embodiments, secondary emission layer 110 may be positioned within focusing cup 106.

FIG. 6 illustrates a further embodiment of the present invention incorporating ferro-electric emission. The primary emission portion 120 of a ferro-electric emitter 118 generally includes a layer of ferro-electric or ceramic-type material 122, such as, for example, PZT (PbZrTiO-based substances) or PLZT (PbLaZrTiO-based substances). In some “bulk disc” embodiments, the layer of ferro-electric material may be approximately 300-500 micrometers in thickness, whereas thin film embodiments may incorporate buffer materials (not shown) and utilize ferro-electric layers having 0.75-1.0 micrometer thicknesses. As shown, ferro-electric layer 122 has a conductive rear electrode 124 on one side and a patterned grid electrode 126 on another side. Grid electrode 126 may be printed to have rows as shown, and each row may be approximately 200 micrometers in width and 5-200 micrometers apart. Both the rear and gate electrodes 124, 126 may be formed of a variety of substances, including platinum and silver. In some embodiments, a buffer layer (not shown) may be deposited between the electrodes 124, 126 and the ferro-electric layer 122.

When a switched or pulsed voltage 128 is applied to rear electrode 124, ferro-electric layer 122 enters an electron emission state. When an accelerating voltage is applied at a thin metal layer or collector 130, the primary stream of electrons 134 is drawn thereto. Preferably, collector 130 is thin enough not to interfere with the flow of primary stream of electrons 134. A secondary emission layer 132 is deposited over collector 130 to amplify primary electron stream 134 and provide a secondary electron stream 136.

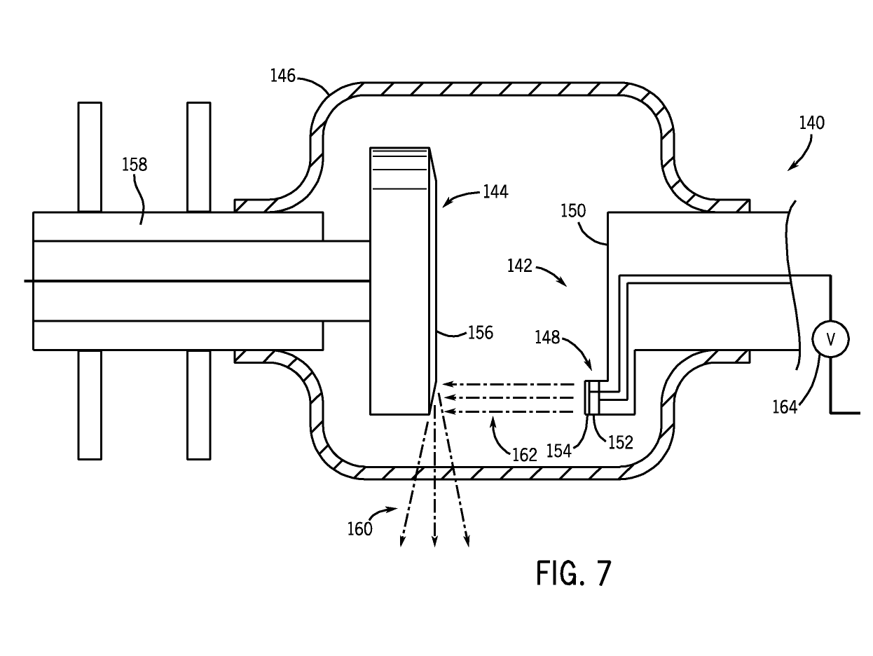

Referring now to FIG. 7, an x-ray generating tube 140, such as for a CT system, is shown. Principally, x-ray tube 140 includes a cathode assembly 142 and an anode assembly 144 encased in a housing 146. Anode assembly 144 includes a rotor 158 configured to turn a rotating anode disc 156, as is known in the art. When struck by an electron current 162 from cathode assembly 142, anode 156 emits an x-ray beam 160 therefrom. Cathode assembly 142 incorporates an electron source 148 positioned in place by a support structure 150. Electron source 148 includes a primary emitter assembly 152 and a secondary emission member 154. Primary emitter assembly 152 may produce a primary electron current as by a field emitter array or a ferro-electric emitter, as described above.

As shown, secondary emission layer 154 is positioned so as to shield primary emitter assembly 152 from the vacuum environment within the x-ray tube. In this regard, secondary emission layer 154 may be manufactured or fitted to securely engage support structure 150. In addition, because of the electron stream amplification characteristics of secondary emission layer 154, x-ray tube 140 is capable of producing x-ray beams 160 with lower input power requirements. Thus, if an x-ray beam of a given intensity is desired, a control voltage 164 may be applied to tube 140 which would otherwise produce an x-ray beam of a lower intensity. For example, if a typical control voltage for a common FEA is 100V to produce an x-ray beam of a given intensity, the control voltage for the present invention to produce the same x-ray beam intensity may be only 10V or less.

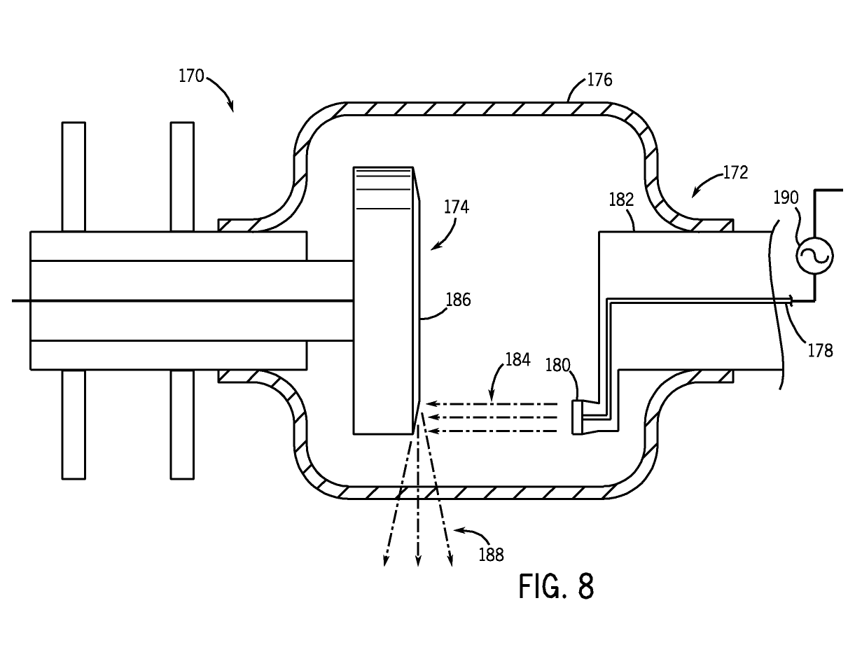

Similarly, FIG. 8 shows an x-ray tube 170 having an anode assembly 174 and a cathode assembly 172 enclosed in a housing 176 wherein the cathode assembly 172 utilizes a thermionic emission filament 178. As described above, a support structure 182 positions filament 178 to direct an electron stream thereof toward anode 186. A secondary emission member 180 is positioned on or adjacent to support structure 182, over filament 178, to shield filament 178 from the tube environment. As such, filament 178 may be composed of less durable materials which are more efficient for electron emission and x-ray generation. Moreover, a controller 190 may be configured to apply a lower current to filament 178 to achieve an electron stream 184 (via secondary emission member 180) sufficient for a desired x-ray beam intensity 188.

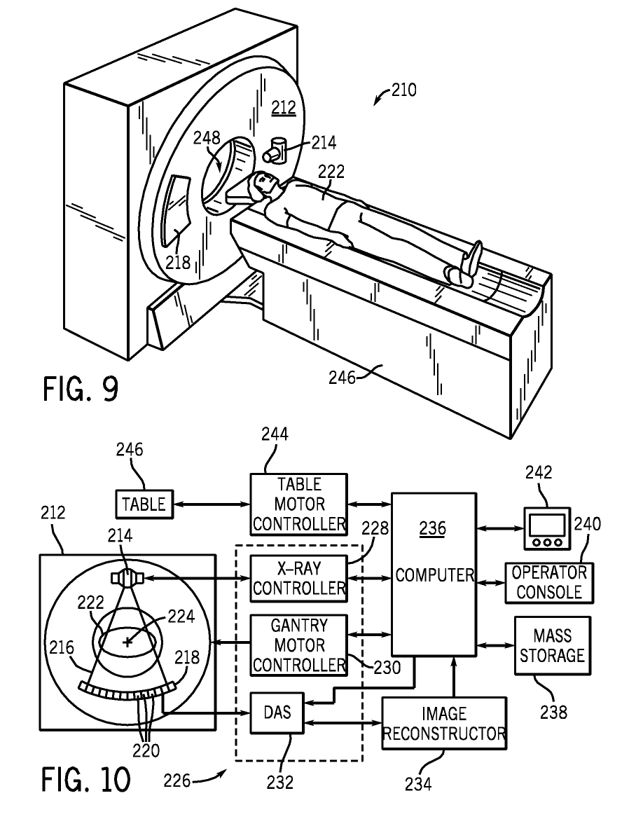

Referring to FIG. 9, a computed tomography (CT) imaging system 210 is shown as including a gantry 212 representative of a “third generation” CT scanner. Gantry 212 has an x-ray source 214 that projects a beam of x-rays 216 toward a detector assembly or collimator 218 on the opposite side of the gantry 212. X-ray source 214 includes an x-ray tube having a cathode constructed as any of the embodiments described above. Referring now to FIG. 10, detector assembly 218 is formed by a plurality of detectors 220 and data acquisition systems (DAS) 232. The plurality of detectors 220 sense the projected x-rays that pass through a medical patient 222, and DAS 232 converts the data to digital signals for subsequent processing. Each detector 220 produces an analog electrical signal that represents the intensity of an impinging x-ray beam and hence the attenuated beam as it passes through the patient 222. During a scan to acquire x-ray projection data, gantry 212 and the components mounted thereon rotate about a center of rotation 224.

Rotation of gantry 212 and the operation of x-ray source 214 are governed by a control mechanism 226 of CT system 210. Control mechanism 226 includes an x-ray controller 228 that provides power, control, and timing signals to x-ray source 214 and a gantry motor controller 230 that controls the rotational speed and position of gantry 12. X-ray controller 228 is preferably programmed to account for the electron stream amplification properties of an x-ray tube of the present invention when determining a voltage or current to apply to produce a desired x-ray beam intensity and timing. Therefore, controller 228 provides computer integration of the lower power requirements of an x-ray generator utilizing secondary emission. An image reconstructor 234 receives sampled and digitized x-ray data from DAS 232 and performs high speed reconstruction. The reconstructed image is applied as an input to a computer 236 which stores the image in a mass storage device 238.

Computer 236 also receives commands and scanning parameters from an operator via console 240 that has some form of operator interface, such as a keyboard, mouse, voice activated controller, or any other suitable input apparatus. An associated display 242 allows the operator to observe the reconstructed image and other data from computer 236. The operator supplied commands and parameters are used by computer 236 to provide control signals and information to DAS 232, x-ray controller 228 and gantry motor controller 230. In addition, computer 236 operates a table motor controller 244 which controls a motorized table 246 to position patient 222 and gantry 212. Particularly, table 246 moves patients 222 through a gantry opening 248 of FIG. 9 in whole or in part.

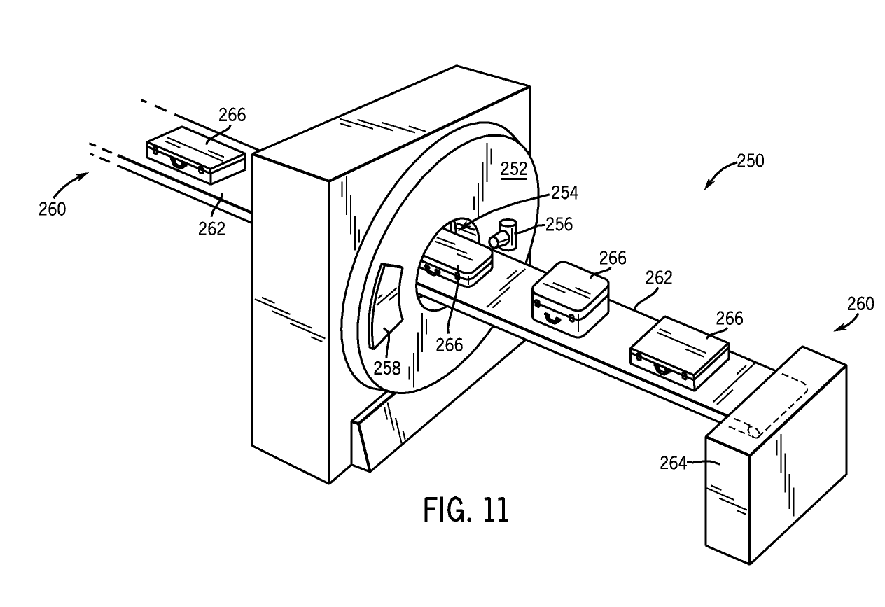

FIG. 11 depicts another implementation of the present invention. A package/baggage inspection system 250 includes a rotatable gantry 252 having an opening 254 therein through which packages or pieces of baggage may pass. The rotatable gantry 252 houses a high frequency electromagnetic energy source 256 as well as a detector assembly 258. The high frequency electromagnetic energy source 256 is configured to utilize secondary electron emission in generating high frequency electromagnetic energy beams, in accordance with the aspects and embodiments of the present invention discussed above. A conveyor system 260 is also provided and includes a conveyor belt 262 supported by structure 264 to automatically and continuously pass packages or baggage pieces 266 through opening 254 to be scanned. Objects 266 are fed through opening 254 by conveyor belt 262, imaging data is then acquired, and the conveyor belt 262 removes the packages 266 from opening 254 in a controlled and continuous manner. As a result, postal inspectors, baggage handlers, and other security personnel may non-invasively inspect the contents of packages 266 for explosives, knives, guns, contraband, etc.

Accordingly, it has been shown that the various aspects and embodiments of the present invention provide for shielded or protected cathode assemblies which require reduced voltage and/or current to produce electron streams sufficient for x-ray generation in a number of operating environments. A technical contribution for the disclosed method and apparatus is that it provides for a computer implemented controller which determines a voltage or current to be applied to an x-ray tube for generating a desired x-ray beam intensity, taking into account the electron stream amplification characteristics thereof.

Therefore, in one embodiment of the present invention, an x-ray generator includes a primary electron source, a controller, a secondary emission component, and an anode. The controller is configured to apply an electrical potential to the primary electron source to cause the primary electron source to emit a primary stream of electrons. The secondary emission component is positioned in the path of the primary stream of electrons and emits a secondary stream of electrons when struck by the primary stream of electrons. The anode is configured to emit x-rays when the secondary stream of electrons strikes the anode.

According to another embodiment of the present invention, a cathode assembly is disclosed. The cathode assembly includes at least one electron emitting member, a secondary emission member, and a controller. The at least one electron emitting member has a first end configured for electron emission and a second end. The secondary emission member is positioned over the first end of the electron emitting member and is separated therefrom. The controller is configured to apply a first voltage to the electron emitting member, thereby generating an electron current from the first end of the electron emitting member. The secondary emission member amplifies this electron current such that it becomes sufficient for generation of x-ray beams.

In accordance with a further embodiment of the present invention, an x-ray tube for an imaging system has a housing that encloses an anode and a cathode. The cathode includes a primary electron emission member and a secondary electron emission member which shields the primary electron emission member. The anode is positioned in an electron path of the cathode and is configured to emit a beam of high-frequency electromagnetic energy conditioned for use in a CT imaging process when a stream of electrons from the cathode impinges thereon.

The present invention has been described in terms of the preferred embodiment, and it is recognized that equivalents, alternatives, and modifications, aside from those expressly stated, are possible and within the scope of the appending claims.

What is claimed is:

1. An x-ray generator comprising:
a primary electron source;
a controller configured to apply an electrical potential to the primary electron source to cause a primary stream of electrons to be emitted from the primary electron source;
a secondary emission component positioned in a path of the primary stream of electrons and configured to emit a secondary stream of electrons when struck by the primary stream of electrons; and
an anode configured to emit x-rays when struck by the secondary stream of electrons.

2. The x-ray generator of claim 1 wherein the controller is further configured to apply the electrical potential to the primary electron source in response to an x-ray generation request, such that the primary stream of electrons alone is insufficient to generate a requested x-ray emission from the anode.

3. The x-ray generator of claim 1 wherein the secondary emission component is designed to emit the secondary stream of electrons having a current greater than a current of the primary stream of electrons.

4. The x-ray generator of claim 1 wherein the secondary emission component is at least partially formed of a diamond-like substance.

5. The x-ray generator of claim 1 wherein the primary electron source is one of a field emitter array and a thermionic emission filament.

6. The x-ray generator of claim 5 wherein the primary electron source is a field emitter array having deposited on a substrate one of Spindt-type cone emitters, carbon nanotubes, inorganic nanowires, and a material having a low work function.

7. The x-ray generator of claim 6 wherein the substrate is opaque.

8. The x-ray generator of claim 1 wherein the secondary emission component forms a gate electrode of the primary electron source.

9. The x-ray generator of claim 8 wherein the controller is connected to apply the electrical potential between the secondary emission component and a substrate of the primary electron source.

10. The x-ray generator of claim 1 wherein the secondary emission component is positioned to shield the primary electron source from stray particles and ion back bombardment and is separated from the primary electron source by at least one of a dielectric material and a vacuum gap.

11. The x-ray generator of claim 1 incorporated into an imaging apparatus.

12. The x-ray generator of claim 1 wherein the primary electron source is at least partially coated with a material having a low work function.

13. A cathode assembly for an x-ray source comprising:
at least one electron emitting member having a first end configured for electron emission and a second end;
a secondary emission member positioned over the first end of the electron emitting member and separated therefrom; and
a controller configured to apply a first voltage to the electron emitting member to generate an electron current from the first end of the electron emitting member that, when amplified by the secondary emission member, is sufficient for generation of x-ray beams.

14. The cathode assembly of claim 13 wherein the electron current from the electron emitting member is insufficient for use in generating an x-ray beam of a pre-selected intensity.

15. The cathode assembly of claim 13 further comprising a substrate layer and a gate layer, and wherein the electron emitting member is positioned between the substrate layer and the gate layer and the secondary emission member is positioned over the gate layer.

16. The cathode assembly of claim 15 wherein the substrate is opaque.

17. The cathode assembly of claim 15 wherein the substrate layer, the gate layer, and the secondary emission member are constructed to have a convex curvature to focus the electron current.

18. The cathode assembly of claim 15 wherein the secondary emission member has a thin metal layer.

19. The cathode assembly of claim 18 wherein the controller is connected to apply the first voltage between the substrate layer and the gate layer and is further configured to apply a second voltage between the substrate layer and the secondary emission member.

20. The cathode assembly of claim 13 wherein the secondary emission member is configured to be a gate electrode for the at least one electron emitting member.

21. The cathode assembly of claim 13 wherein the at least one electron emitting member includes at least one of Spindt-type emitter cones, nanowires, nanotubes, a material having a low work-function, and a thermionic emission filament.

22. The cathode assembly of claim 15 wherein the at least one electron emitting member is at least partially coated with low work function mixed oxide particles.

23. An x-ray tube for an imaging system comprising:
a housing enclosing an anode and a cathode;
the cathode having a primary electron emission member and a secondary electron emission member, wherein the secondary electron emission member shields the primary electron emission member; and
the anode positioned in an electron path of the cathode and configured to emit a beam of high-frequency electromagnetic energy conditioned for use in a CT imaging process when a stream of electrons from the cathode impinges thereon.

24. The x-ray tube of claim 23 wherein the secondary emission member is designed to receive a primary electron current from the primary electron emission member and emit a secondary electron current sufficient to induce from the anode a beam of high-frequency electromagnetic energy of a pre-selected intensity.

25. The x-ray tube of claim 24 wherein the primary electron current alone is insufficient to induce from the anode a beam of high-frequency electromagnetic energy having a pre-selected intensity.

26. The x-ray tube of claim 23 further comprising a plurality of primary electron emission members arranged on an opaque substrate in rows or individually.

27. The x-ray tube of claim 23 wherein the cathode has a convex curvature to focus the stream of electrons therefrom.

28. The x-ray tube of claim 23 incorporated into a CT system, the CT system further comprising:
a rotatable gantry having an opening to receive a subject to be scanned;
a scintillator array having a plurality of scintillator cells wherein each cell is configured to detect the high frequency electromagnetic energy from the anode, passing through the subject;
a photodiode array optically coupled to the scintillator array and comprising a plurality of photodiodes configured to detect light output from a corresponding scintillator cell;
a data acquisition system (DAS) connected to the photodiode array and configured to receive the photodiode outputs; and
an image reconstructor connected to the DAS and configured to reconstruct an image of the subject from the photodiode outputs received by the DAS.

29. The x-ray tube of claim 23 wherein the secondary emission member is positioned over the primary electron emission member to shield the primary electron emission member from stray particles and ion back bombardment.

30. The x-ray tube of claim 23 further comprising a conductive coating about the secondary emission member, and wherein at least one of a gate voltage and a secondary emission voltage is applied thereto.
